(19) United States
(12) Patent Application Publication
(10) Pub. No.: US 2025/0248695 A1
(43) Pub. Date: Aug. 7, 2025

(54) ORAL SAMPLE COLLECTION

(71) Applicant: Able Biomedical Devices, LLC, Seattle, WA (US)

(72) Inventor: O'Beirne Gerrarda, Seattle, WA (US)

(21) Appl. No.: 18/856,708

(22) PCT Filed: Apr. 17, 2023
(86) PCT No.: PCT/US2023/018801

§ 371 Date: Oct. 14, 2024

Related U.S. Application Data
(60) Provisional application No. 63/331,546, filed on Apr. 15, 2022.

(51) Int. Cl. A61B 10/00; A61B 1/24; A61B 5/00; A61C 19/06

(52) U.S. Cl. CPC A61B 10/0051; A61B 1/24; A61C 19/063; A61B 5/0071; A61B 5/0088

(57) ABSTRACT

A sample collecting device is provided, including: a handle having an interface at a first end; and a tip member coupled to the interface, the tip member comprising: a tip body; a lumen passing through the tip body; and a reservoir coupled to a proximal end of the lumen. A sample collecting method is provided, including: positioning a tip member of a device inside a subject's mouth, the device including a handle having an interface at a first end, wherein the tip member is coupled to the interface and the tip member includes: a tip body, a lumen passing through the tip body, and a reservoir coupled to a proximal end of the lumen; guiding the tip member along teeth and/or gums of a subject; and collecting a sample from the subject's mouth in the reservoir of the device.

### CROSS-REFERENCE TO RELATED APPLICATIONS

This application claims the benefit of U.S. Provisional Application No. 63/331,546, filed Apr. 15, 2022, the disclosure of which is incorporated herein by reference in its entirety.

### FIELD

This application relates to oral health, and, more specifically, to devices used for supporting oral health.

### BACKGROUND

Periodontal disease is a chronic inflammatory condition caused by a dysregulated homeostasis between the host immune response and oral bacteria originating in dental plaque. The phenomenon has an impact both locally in the mouth and systemically in the rest of the body. In the uncontrolled diabetic patient, for example, the destructive impact occurs both locally and systemically. The exact synergistic mechanisms that connect periodontal disease and Type 2 diabetes mellitus are unclear, however, it is suspected to be a complex interplay of the innate and adaptive immunity of the host in response to the disseminated microbes and endotoxins from the plaque biofilm. A number of meta-analyses, systematic reviews and studies conducted by the private sector insurance companies concluded that effective management of oral health and gum disease in particular, may bring about a positive impact on management of the chronic diseases that account for the majority of the healthcare burden in the USA, at approximately 80%. Numerous studies have demonstrated links, some bi-directional, between periodontal disease and conditions such as diabetes, heart disease, pregnancy complications and dementia. The benefits go first and foremost to the patient and their health status both orally and systemically. Additionally, tremendous cost savings can be realized with for example, decreased diabetic related health care costs. The end results are improved population health and a lower healthcare economic burden.

Yet, many patients with periodontal disease and systemic conditions such as diabetes do not benefit from effective management of their periodontal disease condition. A number of probable explanations have been suggested. First, it is difficult to consistently remove the plaque biofilm on a daily basis. Toothbrushes and floss have a maximum subgingival penetration of 2-3 mm. Periodontal pockets are much deeper and largely inaccessible. Devices such as the water flossers do reach somewhat further, but the dampening effects of the tissues limit their efficacy. Anti-microbial oral rinses may neutralize the surface bacteria, but have limited impact in the deeper layers of the hydrophobic biofilm matrix where anaerobic bacteria proliferate. Furcations or the “wine glass” shape of implants pose additional problems for access. Second, it is difficult to measure the efficacy of plaque removal or detect early changes heralding the transition from health to disease. This makes it challenging to follow the effectiveness of therapy and modify treatment with any degree of precision. Third, the variety of micro-organisms (the microbiome) within the dental plaque biofilm of patients is highly diverse. Different microbiomes contribute to inflammation potentially having local and systemic effects that may require alternative means of intervention—e.g. application of localized antimicrobial agents or the addition of agents to modify the microenvironment in favor of certain ecological conditions. The interaction of biofilm and the specific host response is central to the disease process. Accordingly, to realize the impact that control of periodontal disease holds to prevent tooth loss, restore oral health and also for the management of systemic conditions such as diabetes, there is an urgent need for tools to (a) accomplish effective daily plaque biofilm removal between dental visits, (b) provide a means to monitor efficacy of plaque removal and deliver feedback to support compliance and engender proper technique, and/or (c) provide a means to easily attain samples of plaque biofilm and gingival cervicular fluid (GCF) for microbiome and inflammatory exudate/transudate analysis that has the potential to inform treatment options both locally and systemically.

### SUMMARY

In accordance with embodiments of the present invention, an oral hygiene device to meet the highly challenging demands for plaque biofilm removal with microbiome and GCF sampling. Various embodiments provide the ability to access deeper pockets, the ability to conform to tooth contours, a camera-guided ability to maneuver into furcations, and sonication to assist in removing plaque. Tips may also be impregnated with anti-microbial agents for microbiome specific, local targeted delivery. A dual feature of the pick member is the ability to collect microbial and GCF samples from the subjects' mouths. In the case of GCF, the sample can be viewed as a liquid biopsy. Samples of plaque biofilm and gingival crevicular fluid (GCF) can be tested via metagenomics, metatranscriptomics, protemics and metabolomics. Strong initial areas of interest may be the presence and profile of biomarkers (e.g., cytokines and other inflammatory markers, enzymes, proteins), certain microflora, the presence of heme or glucose in the sulcus/pocket also the pH of the site. Such devices may be used to improve oral health and potentially have a positive impact systemic health and vis versa.

In accordance with embodiments of the present invention, a sample collecting device is provided, comprising: a handle having an interface at a first end; and a tip member coupled to the interface, the tip member comprising: a tip body; a lumen passing through the tip body; and a reservoir coupled to a proximal end of the lumen.

In accordance with embodiments of the present invention, a sample collecting method is provided, comprising: positioning a tip member of a device inside a subject's mouth, the device comprising a handle having an interface at a first end, wherein the tip member is coupled to the interface and the tip member comprises: a tip body, a lumen passing through the tip body, and a reservoir coupled to a proximal end of the lumen; guiding the tip member along teeth and/or gums of a subject; and collecting a sample from the subject's mouth in the reservoir of the device.

Still other embodiments of the present invention will become readily apparent to those skilled in the art from the following detailed description, which describes embodiments illustrating various examples of the invention. As will be realized, the invention is capable of other and different embodiments and its several details are capable of modifications in various respects, all without departing from the spirit and the scope of the present invention.

### BRIEF DESCRIPTION OF DRAWINGS

FIG. 1A illustrates an angled view of an oral hygiene device, in accordance with various aspects of the present disclosure.

FIG. 1B illustrates a side view of the oral hygiene device, in accordance with various aspects of the present disclosure.

FIG. 1C illustrates a front view of the oral hygiene device, in accordance with various aspects of the present disclosure.

FIG. 1D shows a perspective view of the bottom of the device, in accordance with various aspects of the present disclosure.

FIG. 2A is an enlarged view of the head portion of the device, in accordance with various aspects of the present disclosure.

FIG. 2B shows the use of a tip member to collect samples from a subject's mouth and/or deliver agents to the subject's mouth, in accordance with various aspects of the present disclosure.

FIG. 3A shows a cross-section of a tip member, in accordance with various aspects of the present disclosure.

FIG. 3B is a perspective view of a tip member, in accordance with various aspects of the present disclosure.

FIG. 4 shows a cross-section of a tip member having a compressible diaphragm, in accordance with various aspects of the present disclosure.

FIGS. 5A-5C illustrate another embodiment of a head portion and tip member, in accordance with various aspects of the present disclosure.

FIG. 6 illustrates a cross section of the head portion, in accordance with various aspects of the present disclosure.

FIGS. 7A-7B illustrate another embodiment of a head portion and tip member, in accordance with various aspects of the present disclosure.

FIGS. 8A-8B illustrate multiple camera alignments with a tip member, in accordance with various aspects of the present disclosure.

### DETAILED DESCRIPTION

In the following description, reference is made to the accompanying drawings that illustrate several embodiments of the present disclosure. It is to be understood that other embodiments may be utilized and system or process changes may be made without departing from the spirit and scope of the present disclosure. The following detailed description is not to be taken in a limiting sense, and the scope of the embodiments of the present invention is defined only by the claims of the issued patent. It is to be understood that drawings are not necessarily drawn to scale.

Currently paper strips are used to collect GCF and a sterile pointed stainless steel instrument is used to collect biofilms. This technology (paper strips/points) has seen no further significant advancement for years. It would be desirable to provide tools that specifically target collection of these biomaterials and incorporate containment systems for sterile transportation from the collection point to the point of analysis. Micropipettes are also used to collect GCF and saliva, however without the incorporation of a suction system, this process is slow (taking up to 30 minutes) and there is the potential for added trauma to the site, with probable interference with the results of the sample.

Paper strips are easily damaged and contamination in transportation is a considerable risk. In the case of collection of biofilms, removal of the biomaterial from tooth, root or other oral surface via a random sterile instrument and insertion into a random vial with a liquid vehicle, such as sterile saline, lacks standardization. Alteration and/or contamination of the compositional material is a significant risk. Described herein are embodiments of tools for collection and storage of GCF, saliva, biofilms, and samples from a subject's mouth so that the integrity of the samples is protected, allowing for the collection of meaningful data.

In the case of the oral diseases, periodontal disease and caries, the disease front is often hard to reach as it either below the gum or between the teeth. In accordance with embodiments of the present invention, a tip member is provided that can deliver therapeutic agents directly, through release of molecules from the surface of the tip member, a fluid with an agent that is soaked into the surface, as with a hydrogel, or contained within a reservoir on the tip member, that can be emptied as part of creation of a vacuum, spring release, or an electronic activation system across the surfaces. Iontophoresis is an active delivery method whereby physiologically acceptable electric currents move charged agents across the sulcus/pocket lining by a potential gradient. This may be used instead of or in conjunction with a passive method, such as nano-emulsification that could increase molecular permeability. Iontophoresis can be used to deliver many different molecules such as antimicrobials, fluoride, pain medications both non-steroidal and opioids, anaesthetics, vitamins and even cancer drugs. Various polymer systems can be used including silver. Electrophoretic drug delivery systems are also under investigation.

Use of conducting polymers in a tip member may facilitate the incorporation of point-of-care (POC) sensors. A biosensor may utilize a combination of the following stages:

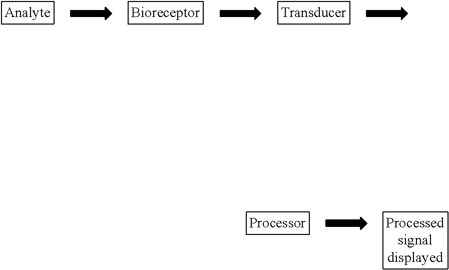

Analytes could be biomarkers such as TNFα, HIF-1α or other cytokines/mediators (IL1β, IL6, matrix metalloproteins (MMP's), interferon, prostaglandins), host derived enzymes, microbial or host tissue components and metabolites including DNA/RNA, biomimetic materials or breakdown elements such as minerals, molecules such as hemoglobin or glucose. Bioreceptors recognize the analyte and could be antibodies, enzymes, cells, aptamers (nucleic acids or microbial strains) or some nanoparticles that produce a signal (e.g., pH change, electron transfer, mass change, heat change, gas or specific ion release). The transducer converts energy from one form to another, producing a signal that has been referenced. The transducer is central and can be, e.g., electrochemical, optical, electronic, gravimetric or thermal. The transduced signal is processed, amplified, and prepared for display.

The sensor/bioreceptor on the tip member may recognize the analyte, thereby producing a signal that goes to a transducer and processor on the device, which detects a change in potential. The device may then connect (e.g., using wireless network communications such as Wi-Fi) to a database or possibly directly, allowing for correlation with standardized readings. The output could reveal diagnostic findings informing the user about the current state of health or disease.

Alternately, the entire tip member can be shipped in a sealed container to a laboratory for processing. Various bioreceptors and transducers would be available to analyze the sample at the laboratory.

As our understanding of what constitutes health and disease evolves and our knowledge of the molecules (biomarkers, etc.) that guide or indicate the stages of this transition continue to develop, it is desirable to provide precise tools for collecting the biomaterials under discovery and/or deliver impactful agents to selective sites in the mouth. POC products may use diagnostic markers to indicate and/or calibrate the active degree process and point to use of targeted therapeutic options.

In accordance with embodiments of the present invention, a tip member is provided for use in the diagnosis and/or management of oral diseases, particularly periodontal disease and caries. The tip member may be used to collect biological material(s) (e.g., proteins, enzymes, blood components such as hemoglobin, immune molecules, micro-organisms, cellular elements, electrolytes and organic compounds such as glucose, genetic material (DNA)), and/or dispense agents to have a biologically positive effect. For example, the tip member may be used to collect oral fluids, e.g., gingival crevicular fluid (GCF), saliva, and oral biofilms-together or independently for analysis. The tip member can also be used to collect oral biofilms particularly from above and or below the gum tissues around teeth and or dental implants. For example, the tip member may be used to collect the gingival crevicular fluid from the sulcus around a tooth and/or any overflow that is at or above the gumline.

The tip member can also carry an active agent to the tissues of the tooth and gums to modify, arrest, or reverse a disease process, and sometimes strengthen the tissues against damage from further insult. In some embodiments, the tip member may also be used to disperse or deliver active agents to abort the disease process, restore the dental site to health and or strengthen the tooth surface (e.g., antimicrobials, fluoride, arginine and other amino acids, essential oils, sugar alcohols such as xylitol and erythritol, peroxides, synthetic bacteria, sodium hypochlorite, sodium chlorate, hypochlorous acid, probiotics and prebiotics, chlorine dioxide, or engineered molecules that interfere with either biofilm formation/growth or with the immune response and/or the complement system).

The gingival crevice is a porous portal to the rest of the body and may serve as a delivery method for certain agents with systemic implications. In accordance with embodiments of the present invention, the tip member can be used to carry agents to facilitate entry to the vascular system and/or the interstitial tissues to bring about a desired systemic effect.

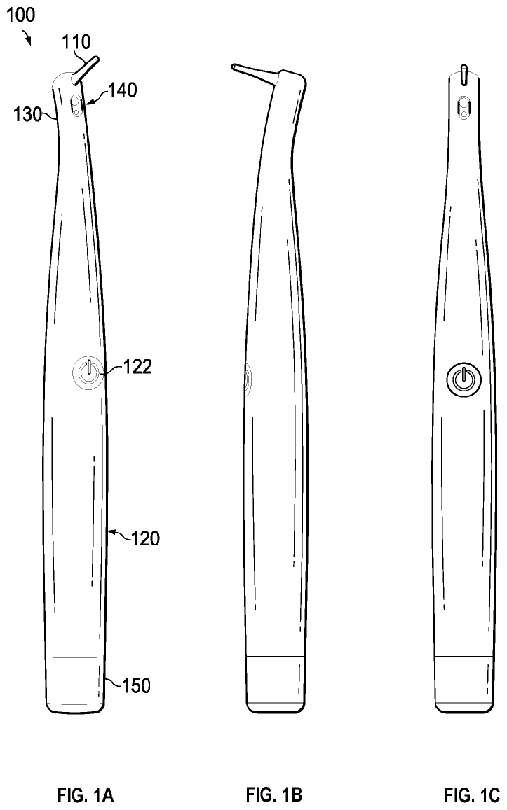

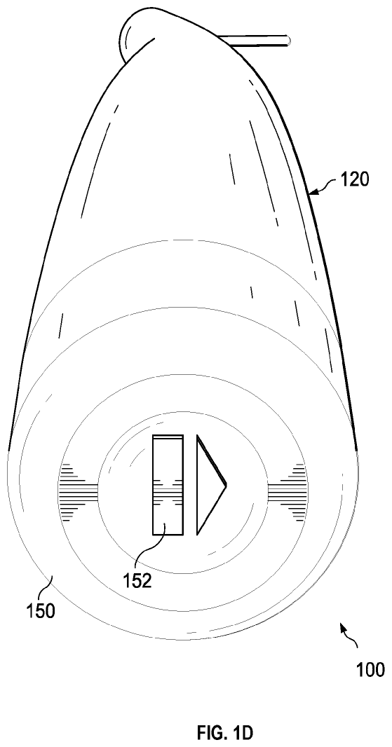

FIGS. 1A-1D illustrate various views of an oral hygiene device 100 that may be used with a tip member 300, in accordance with embodiments of the present invention. Examples of this device are described in more detail in U.S. Pat. No. 11,173,018, issued Nov. 16, 2021, the contents of which are incorporated herein in their entirety.

FIG. 1A shows an angled view of the device 100, FIG. 1B shows a side view of the device 100, FIG. 1C shows a front view of the device 100, and FIG. 1D shows a perspective view of the bottom of the device 100. In this embodiment, the oral hygiene device 100 is a powered toothpick handset utilizing sonic vibration of a replaceable pointed pick head 110. The pick head 110 may be used for cleaning teeth and gums.

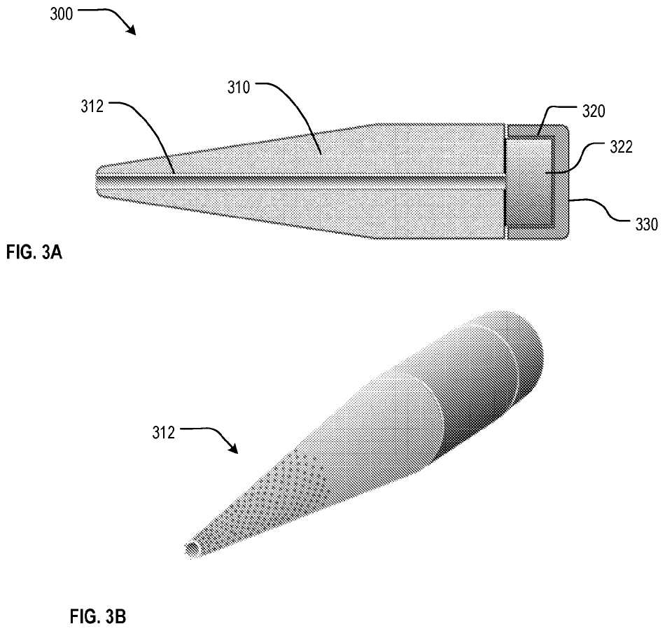

In accordance with embodiments of the present invention, the pick head 110 shown in FIGS. 1A-1D may be removed and replaced with a tip member 300, shown in FIGS. 3A-3B. This tip member may be used for sampling and/or delivery of agents.

The device 100 includes a handle portion 120 which is gripped by the user when utilizing the device. A head portion 130 is provided at a distal end of the device 100 and an inductive charging portion 150 is provided at a proximal end of the device 100. The dimensions of the device 100 may vary, but in one embodiment, the device 100 is approximately 140 mm tall, 12 mm wide at the inductive charging portion 150, 5 mm wide at the head portion 130, and the tip member 300 is approximately 10 mm long. The head portion 130 includes an interface 135 for coupling with the tip member 300. The interface 135 securely holds the tip member 300 in place while the user is operating the device 100. The tip member 300 includes a pointed pick member 111, which may comprise any material suitable for use in cleaning teeth and gums.

The device 100 may include a rechargeable battery and charger circuitry. The battery may be recharged through an electrical connector or via a wireless charging interface (e.g., the inductive charging portion 150). The inductive charging portion 150 includes a secondary coil for coupling with an electromagnetic field generated by a primary coil in a charger base (not shown), similar to the way in which conventional electric toothbrushes are charged.

As seen in FIG. 1D, the bottom of the device 100 may include an eject button 152, which, when depressed by the user, ejects the tip member 300 from the interface 135. In some examples, interface 135 may be a press-fit connection which is effective to securely hold tip member 300 to device 100. Depressing eject button 152 may cause a release mechanism to produce a force along the axis of tip member 300 (e.g., along the long axis of tip member 300 which extends along tip member 300 from device 100 to the exposed tip of pick member 111), the force being directed away from device 100 along the axis of tip member 300, such that the force decouples tip member 300 from interface 135. In some examples, depressing eject button 152 may cause a dilation of an enclosure which at least partially surrounds and securely holds a portion of tip member 300 inside of device 100. Dilation of the enclosure may allow for tip member 300 to be easily removed from device 100. In some examples, when the enclosure is dilated by depressing eject button 152, orienting device 100 in such a way that the long axis of tip member 300 faces downward, may allow tip member 300 to be removed by the force of gravity without requiring a user to apply a pulling force to tip member 300. In another example, when the enclosure is dilated by depressing eject button 152, a mechanism such as a piston, flexible diaphragm, or other structure disposed within device 100 and coupled to eject button 152 may apply a force to the portion of tip member 300 which is disposed inside of device 100. Such a force may be directed away from device 100 along the axis of tip member 300, and may eject tip member 300 from device 100 while the enclosure is dilated as a result of the depression of eject button 152. Embodiments described above where the tip member 300 may be removed without requiring a user to touch the tip member 300 may be advantageous, particularly in examples where the tip member is to be sent for microbial or fluid analysis, where human handling of the tip member may otherwise contaminate the collected sample.

In accordance with other embodiments, other mechanisms for retaining and releasing the tip member from the device may be used. For example, a wooden tip member may have a threaded base that can screw into a corresponding threaded interface in the device.

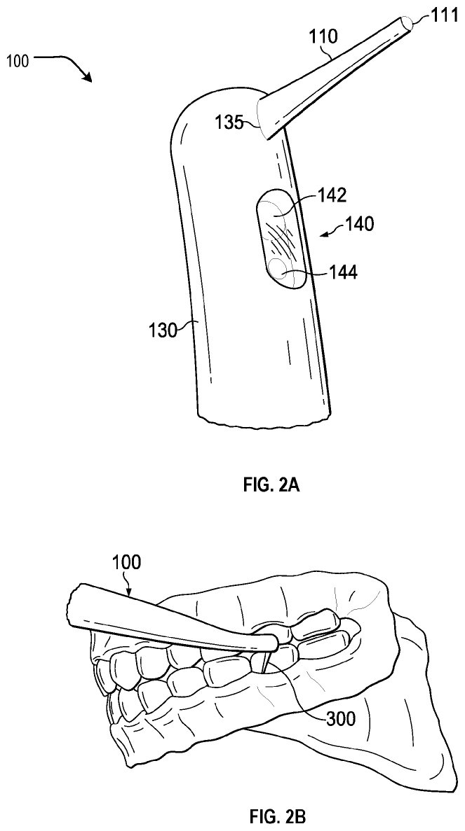

FIG. 2A is an enlarged view of the head portion 130. In some embodiments, the device 100 includes an image capture device 140 for capturing images of area surrounding the pick head 110 or tip member 300, such as portions of the subject's mouth, teeth, and gums during operation of the device 100. The image capture device 140 includes a lens 142 for a digital camera module contained within the device 100, and a light source 144 (e.g., an LED light) for illuminating the area to be imaged. The image capture device 140 may capture still images or video of the area surrounding the tip member 300, and may store this data in a memory (e.g., located inside of device 100) for later analysis and/or transmission to a memory in charger base or other computing device, or may transmit the images and/or video in real-time over a network, (e.g., a local area network or wide area network (WAN)) to another computing device, such as a computer operated by the subject's health care provider. The light source 144 may comprise a white LED, or may produce a different color to accentuate the features being observed. For example, in fluorescent based detection, biometabolites such as porphyrins, are found in all living cells including bacteria. They absorb certain wavelengths of light alone or in combination with other agents. Excitation of the selected substances results in emission of fluorescence which may subsequently need to be post processed in a camera filter or via software and pixel segmentation options. The light source 144 may generate ultraviolet light, which may be used in conjunction with fluorescent markers, stains, and/or other plaque-disclosing tablets. In the illustrated embodiment, the lens 142 and light source 144 are positioned adjacent to each other and aligned so as to be directed to the distal end of the tip member 300. In other embodiments, different configurations may be used, such as, e.g., a lens surrounded by several light sources arranged around a circumference of the lens. In some other examples, head portion 130 and/or device 100, more generally, may include a light source 144 without a camera module or other imaging sensor to allow for illumination of a user or subject's mouth without capturing images, or may include an imaging sensor without a light source. Image capture and visualization may be useful to identify and/or reference the exact location of a sampled site or where a therapeutic agent has been delivered.

In some embodiments, the tip member 300 may include gradations or other markings to provide a visual indication of the depth that the tip member 300 is inserted, e.g., beneath the gums, between the subject's teeth, and/or within pockets or furcations on the subject's teeth and/or implants.

### Tip Member

In accordance with embodiments of the present invention, the device 100 may be used with a sampling tip member 300 to collect samples from a subject's mouth, as shown in FIG. 2B. The sampling tip member 300 may be guided along a subject's teeth, slightly beneath the gum line of the subject's teeth, between the subject's teeth, and/or within pockets or furcations on the subject's teeth and/or implants during a sample collection session. In accordance with embodiments of the present invention, the materials and structures for the sampling tip member 300 will serve the dual purpose of sampling biofilm-based bacteria and gingival crevicular fluid (GCF). In some embodiments, the tip member 300 may employ surface features and be constructed of a composite or hybrid of several materials. An example of such a structure is shown in FIGS. 3A-3B. In this design, a cellulose structure 310 with a micro- or nano-patterned surface region 312 may be adapted to tooth/root/implant surface and collect bacterial plaque present on the tooth. The patterned surface region may serve to increase the potential biofilm sample volume that can be collected on the surface of the cellulose. This increased sample volume may enhance the reliability (sensitivity and specificity) of downstream biological analysis of the sampled biofilm. A user may sweep the patterned surface region 312 across the tooth, root, or implant surface to collect biofilm samples. The size of the pores patterned surface region 312 could be, e.g., in the millimeter, micrometer, or nanometer scales.

Within the cellulose/wood/other material component, a lumen is formed in which a polymer micro-capillary tube 312 or bundle of tubes is provided. The capillary tube 312 may be used to gather and rapidly transport a sample of GCF to a highly absorbent wicking fluid reservoir 320 contained within a cap 330. The wick material 322 acts to draw fluid from the capillary tube 312 and store a large sample volume of GCF in the sterile reservoir 320 for use in downstream biological analysis. The wick material 322 may comprise any suitable material for collection of biosamples, such as, e.g., poly lactic acid, bioplastic, biocomposites, bamboo, paper, or flax.

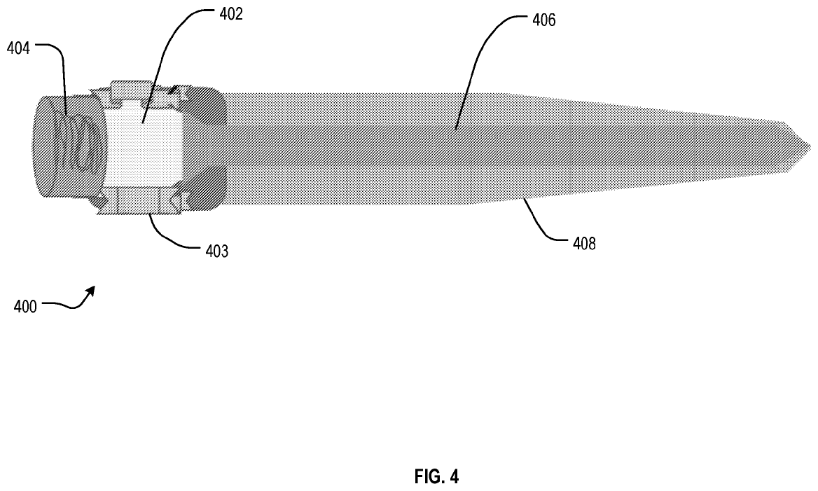

In some embodiments, biofilm or GCF may be collected on the surface of or within the material of the tip member. In some embodiments, it may be desirable to retrieve the sample faster so as to avoid traumatizing the sample site. In some embodiments, a mechanism may be used to create a vacuum or suction in the lumen in order to more effectively draw the sample into the reservoir 320, either in addition to or in place of the capillary action caused by capillary tube 312. This may be achieved using a chamber or diaphragm that can be compressed and released in order to generate the desired suction in the lumen. FIG. 4 shows a cross-section of a tip member 400 having a compressible diaphragm/reservoir 402. The walls of the reservoir 402 may comprise rubber or other elastic material. In some embodiments, the walls of the reservoir 402 could contain springs 404 that are depressible such that a vacuum is created once released, again drawing the sample up the capillary tubing 406 with a greater vacuum suction force. In some embodiments, removable wicking paper or other absorbent material may be provided inside the capillary tubing 406 to absorb the sample. In some embodiments, the surface 408 of the

In some embodiments, the tip member 300 may comprise wood, which may include, e.g., approximately 40-50% cellulose content. Wood is softer than dentin (root surface), and can act as a vehicle and absorb and release agents such as essential oils, amino acids, salts such as fluoride, chloride, peroxide, iodine, hypochlorous acid, nitric acid, tree sugars such as xylitol, erythritol and stevia and other agents including synthetic agents (proteins, amino acids such as arginine, synthetic bacteria) that could favorably impact the oral biome/tooth or root surface. In a moist environment the cellulose material of the tip member 300 could conform to the curvatures of the tooth/root/implant. In other embodiments, the tip member 300 may comprise elastomers or bioplastics, ideally sustainable and biodegradable and may have a surface 408 composed of a hydrogel that could potentially dissolve and thereby release an agent into the tissues for positive effect.

The tip member 300 may include a handle interface portion (not shown) which is disposed inside of device 100 and which engages with interface 135 may have one or more flanges and/or recessed portions which may securely engage with the enclosure of interface 135, described above. In some other examples, interface 135 may include a magnet which is effective to securely hold tip member 300 to device 100 in conjunction with the size and shape of the portion of tip member 300 which fits into device 100, and the size and shape of the portion of interface 135 which is configured to receive tip member 300. In examples where interface 135 includes a magnet, tip member 300 may comprise a ferrous material positioned adjacent to the interface 135 when the tip member 300 is engaged with the interface 135.

After the tip member 300 has been used to collect the desired samples from the subject's mouth, the tip member 300 may be detached so that the samples may be analyzed. In some embodiments, the entire tip member 300 may be immediately analyzed or be placed into a sterile container for transportation to the location for the downstream biological analysis. In other embodiments, the cap 330 containing the wick material or fluid collected 322 in the (wicking fluid) reservoir 320 may be detached from the rest of the tip member 300 and placed in a sterile container for analysis.

A variety of methods may be used to extract the sample from the wicking fluid reservoir 320. For example, the reservoir could be emptied using micropipettes for analysis via an immunoassay (ELISA). In other examples, the wick material 322 may be steam washed and placed in suspension in phosphate buffered saline. The suspension can be then analyzed to measure total genetic material or to look at viable cells, molecules, enzymes, metabolic byproducts of the host or the microflora. Pyrosequencing may be employed to identify species. In order to analyze biofilm volume, optical coherence tomography may be used. In order to analyze biofilm cell density, confocal laser scanning microscopy may be used. A scanning electron microscope (SEM) may be used to image the surface of the tip member 300 covered in biofilm.

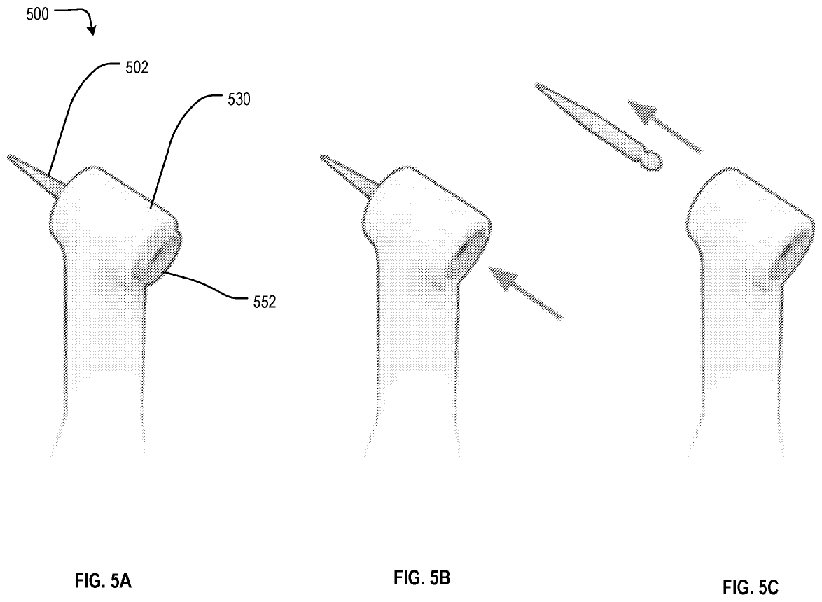

FIGS. 5A-5C illustrate a device 500 including a head portion 530 which securely retains a tip member 502, in accordance with other embodiments of the present invention. FIG. 5A shows the head portion 530 retaining the tip member 502 in the operational state that the device 500 would be used for collecting samples, as described above. FIG. 5B shows eject button 552 in the depressed state, whereby the tip member 502 is released from the head portion 530. FIG. 5C shows the released tip member 502 being withdrawn from the head portion 530.

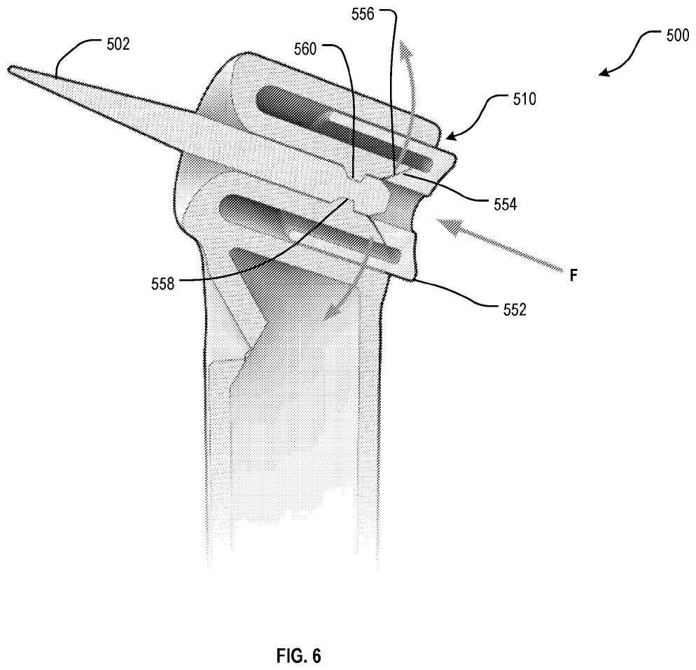

FIG. 6 shows a cross section of the head portion 530 illustrating a release mechanism 510 for the tip member 502, in accordance with embodiments of the present invention. The proximal end of the tip member 502 includes an annular groove 558. When in the operational state to collect samples, the groove 558 engages a ridge 560 on an interior flange 556 of the head portion 530. To release the tip member 502, a force F is applied to the eject button 552, which causes a ramped surface of an eject flange 554 to press against a ramped surface of the interior flange 556. This causes the interior flange 556 to expand such that the ridge 560 is no longer retained by the groove 560, and the tip member 502 can be withdrawn from the head portion 530.

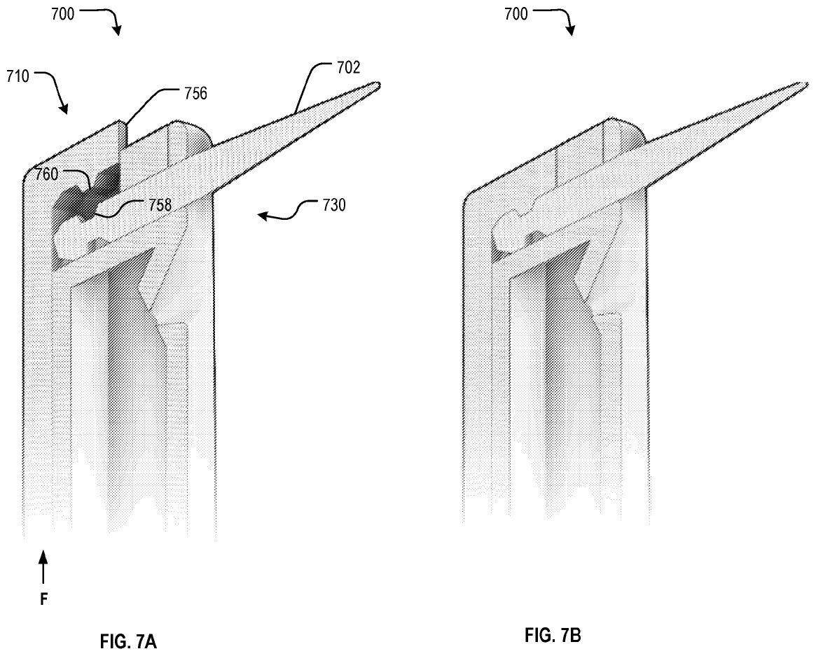

FIGS. 7A-7B illustrate a device 700 including a release mechanism 710, in accordance with other embodiments of the present invention. The proximal end of the tip member 702 includes an annular groove 558. When in the operational state to collect samples, the groove 758 engages a ridge 760 on sliding flange 756 of the head portion 730. To release the tip member 702, a force F is applied to an eject button (not shown), which causes the sliding flange 756 to translate upwards, thereby disengaging the groove 758 from the ridge 760 and releasing the tip member 702 from the device 700. FIG. 7A depicts the release mechanism 710 in the open position, whereby the tip member 702 can be inserted or removed from the device 700, and FIG. 7B depicts the release mechanism 710 in the closed position, whereby the tip member 702 is securely retained for use in a subject's mouth.

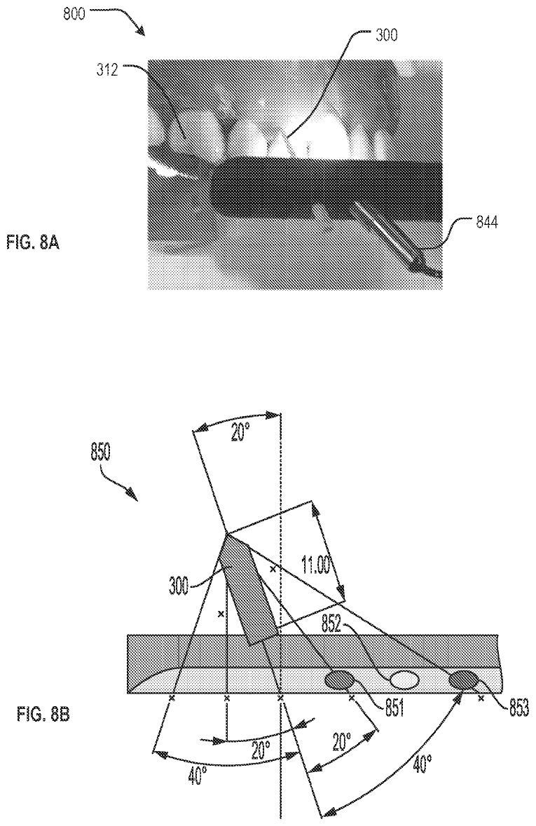

FIGS. 8A-8B illustrate multiple camera alignments with a tip member 300, in accordance with various aspects of the present disclosure. A light source 844 is positioned adjacent to the tip member 300 such that the light projecting from the light source 844 is directed to the location of the distal end of the tip member 300.

In FIG. 8B, various exemplary angles for the tip member 300 and light source 844 are shown. The tip member 300 may be tilted such that a central axis of the tip member 300 is approximately 20° from a line orthogonal to the central axis of the handle of the device. A first position 851 for the light source 844 is set such that the light is directed to the distal end of the tip member 300 at an angle of approximately 20° from the central axis of the tip member 300, a second position 852 for the light source 844 is set such that the light is directed to the distal end of the tip member 300 at an angle of approximately 30° from the central axis of the tip member 300, and a third position 853 for the light source 844 is set such that the light is directed to the distal end of the tip member 300 at an angle of approximately 40° from the central axis of the tip member 300. These configurations are merely exemplary and different positions and angles may be used.

Biosensors may be used to detect host tissue breakdown products, break down products from the microflora, host derived enzymes or their inhibitors or inflammatory mediators or host response modifiers. The samples may be sent to dedicated laboratories for analysis. In other embodiments, POC sensors may be used so that specific analytes will be detected by bioreceptors and passed through a transducer and processed accordingly. Nanotechnology is rapidly developing in this area with miniaturized bioreceptors and transducers and will move us more towards POC sensors for liquid biopsies (GCF and saliva) in particular.

The device may include a camera, gyroscope, and/or accelerometer for determining the position of the tip member within the mouth. In some embodiments, the tip member 300 may have visual reference markings (not shown), which may be used in conjunction with data recorded by an integrated digital camera module to determine various characteristics such as the relative position of the tip member 300 in a subject's mouth or the depth of a pocket found in the user's teeth.

The designs described herein are merely examples of the approach and configurations of the tip members in accordance with various embodiments of the present invention. These tips may be composites of purpose-selected materials, employing micro or nano features, to provide excellent sampling performance for both GCF and bacterial biofilms. It should be appreciated that the geometry of the prototype tip members can be varied depending on the specific sample collection site to be addressed and ease of use for the healthcare provider.

While the invention has been described in terms of particular embodiments and illustrative figures, those of ordinary skill in the art will recognize that the invention is not limited to the embodiments or figures described. For example, in various embodiments described above, the tip member is used as a sampling tip member for collecting samples from a subject's mouth. In other embodiments, the tip member can be used to deliver agents to the subject's mouth, either in addition to collecting samples or solely for agent delivery.

In some embodiments, the device 100 may be a multi-purpose device used with both a vibrating pick head 110 and a tip member 300. In other embodiments, the tip member may be used with a dedicated device/handle. The device may include a camera and light source, or may be a sample handle for releasably holding the tip member during collection with or without a light source and/or camera. The device may be modified to accommodate use in research facilities.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one,” “at least one” or “one or more.” Unless otherwise required by context, singular terms used herein shall include pluralities and plural terms shall include the singular.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While specific embodiments and examples for the disclosure are described herein for illustrative purposes, various equivalent modifications are possible within the scope of the disclosure, as those skilled in the relevant art will recognize. Such modifications may include, but are not limited to, changes in the dimensions and/or the materials shown in the disclosed embodiments.

Specific elements of any embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Therefore, it should be understood that the invention can be practiced with modification and alteration within the spirit and scope of the appended claims. The description is not intended to be exhaustive or to limit the invention to the precise form disclosed. It should be understood that the invention can be practiced with modification and alteration and that the invention be limited only by the claims and the equivalents thereof.

1-23. (canceled)

24. A sample collecting device, comprising:
a handle having an interface at a first end; and
a tip member releasably coupled to the interface, the tip member comprising:
a tip body;
a lumen passing through the tip body; and
a reservoir coupled to a proximal end of the lumen.

25. The device according to claim 24, wherein:
the tip member comprises a proximal end coupled to the interface and a pointed distal end having an aperture, wherein the lumen extends from the reservoir to the aperture of the pointed distal end of the tip member.

26. The device according to claim 24, wherein:
the handle comprises a light source configured to direct light to a region surrounding a distal end of the lumen.

27. The device according to claim 24, wherein:
the handle comprises an imaging device configured to capture images of a region surrounding a distal end of the lumen.

28. The device according to claim 24, wherein:
the reservoir is detachable from the tip body.

29. The device according to claim 24, wherein:
the reservoir comprises a compressible chamber.

30. The device according to claim 24, wherein:
the reservoir comprises a spring configured to expand a volume of the reservoir.

31. The device according to claim 24, wherein:
the reservoir comprises an absorbent material.

32. The device according to claim 24, further comprising:
an absorbent material provided in the lumen.

33. The device according to claim 32, wherein:
the absorbent material comprises a removable wicking paper.

34. The device according to claim 24, wherein:
an exterior surface of the tip member includes a plurality of regularly-spaced markings.

35. The device according to claim 24, further comprising:
a therapeutic agent provided on an exterior surface of the tip member.

36. The device according to claim 35, wherein:
the therapeutic agent comprises one or more of: a probiotic treatment; molecules effective to block bacterial functions; molecules effective to block metabolic byproducts that fuel inflammation; antibodies or molecules that block a surface antigen or receptor; an enzyme treatment effective to slow or prevent bacterial attachment; non-pathogenic organisms effective to slow, outcompete, or prevent attachment of pathogenic biofilms; molecules effective to down-regulate an inflammatory response in teeth or gums; or molecules effective to elevate pH in a subject's mouth.

37. The device according to claim 35, wherein:
the therapeutic agent comprises one or more of: hydrogen peroxide, xylitol, fluoride, ginger, pineapple, cranberry, sodium hypochlorite, sodium chlorate, chlorine dioxide, chlorhexidine, essential oils, spearmint oil, tea tree oil, arginine, or quinone.

38. The device according to claim 24, wherein:
the tip body comprises wood or a cellulose material.

39. The device according to claim 24, wherein:
the tip body comprises a material softer than 3 Mohs on the Mohs hardness scale.

40. A sample collecting method, comprising:
positioning a tip member of a device inside a subject's mouth, the device comprising a handle having an interface at a first end, wherein the tip member is releasably coupled to the interface and the tip member comprises: a tip body, a lumen passing through the tip body, and a reservoir coupled to a proximal end of the lumen;
guiding the tip member along teeth and/or gums of a subject; and
collecting a sample from the subject's mouth in the reservoir of the device.

41. The method according to claim 40, wherein:
the tip body comprises a pointed tip end having an aperture; and
the lumen extends from the reservoir to the aperture of the pointed tip end.

42. The method according to claim 40, wherein:
the collecting the sample from the subject's mouth comprises collecting one or more of: a microbial sample, a fluid sample, a biofilm sample, or a gingival crevicular fluid sample.

43. The method according to claim 40, further comprising:
dispensing a therapeutic agent from an exterior surface of the tip member to the subject's mouth.

44. The method according to claim 40, further comprising:
dispensing a treatment effective to enhance one or more of a biofilm management or inflammatory process management from the tip member to one or more of the teeth or gums of the subject.

45. The method according to claim 40, further comprising:
dispensing a therapeutic agent comprising one or more of: a probiotic treatment; molecules effective to block bacterial functions; molecules effective to block metabolic byproducts that fuel inflammation; antibodies or molecules that block a surface antigen or receptor; an enzyme treatment effective to slow or prevent bacterial attachment; non-pathogenic organisms effective to slow, outcompete, or prevent attachment of pathogenic biofilms; molecules effective to down-regulate an inflammatory response in teeth or gums; or molecules effective to elevate pH in a subject's mouth.

46. The method according to claim 40, further comprising:
analyzing the sample from the subject's mouth.
